(19) United States
(12) Patent Application Publication
(10) Pub. No.: US 2024/0423614 A1
(43) Pub. Date: Dec. 26, 2024

(54) SYSTEMS AND METHODS FOR EMBEDDING MEDICAL ADDITIVES INTO BIOABSORBABLE MATERIALS

(71) Applicant: CILAG GMBH INTERNATIONAL, Zug (CH)

(72) Inventors: Brody FROST, Fairfield, OH (US); Madelaine FRANZONI, Cincinnati, OH (US); Michael J. VENDELY, Lebanon, OH (US); Peyton HOPSON, Cincinnati, OH (US)

(21) Appl. No.: 18/485,161

(22) Filed: Oct. 11, 2023

Related U.S. Application Data
(60) Provisional application No. 63/522,660, filed on Jun. 22, 2023.

(51) Int. Cl. A61B 17/068; A61L 17/00; A61L 17/10

(52) U.S. Cl. CPC A61B 17/068; A61L 17/005; A61L 17/10; A61B 2017/00004; A61L 2300/604

(57) ABSTRACT

The disclosed technology includes a method for embedding medical additives into a bioabsorbable material. The method may include chemically reacting one or more functional groups of a bioabsorbable material with one or more medical additives. The bioabsorbable material may be configured to be placed inside a body of a human. The one or more medical additives may be configured to remain chemically bonded to a foam structure of the bioabsorbable material until a biodegradation of the bioabsorbable material.

### CROSS-REFERENCE TO RELATED APPLICATIONS

This application claims priority under 35 U.S.C. § 119 to U.S. Provisional Patent Application No. 63/522,660, filed Jun. 22, 2023, the entire contents of which are fully incorporated herein by reference.

### FIELD

The present invention relates generally to systems and methods for embedding medical additives into bioabsorbable materials.

### BACKGROUND

Surgical staplers are used in surgical procedures to close openings in tissue, blood vessels, ducts, shunts, or other objects or body parts involved in the particular procedure. The openings can be naturally occurring, such as passageways in blood vessels or an internal organ like the stomach, or they can be formed by the surgeon during a surgical procedure, such as by puncturing tissue or blood vessels to form a bypass or an anastomosis, or by cutting tissue during a stapling procedure.

Most staplers have a handle (some of which are directly user operable, others of which are operable by a user via a robotic interface) with an elongate shaft extending from the handle and having a pair of movable opposed jaws formed on an end thereof for holding and forming staples therebetween. The staples are typically contained in a staple cartridge, which can house multiple rows of staples and is often disposed in one of the two jaws for ejection of the staples to the surgical site. In use, the jaws are positioned so that the object to be stapled is disposed between the jaws, and staples are ejected and formed when the jaws are closed, and the device is actuated. Some staplers include a knife configured to travel between rows of staples in the staple cartridge to longitudinally cut and/or open the stapled tissue between the stapled rows.

### SUMMARY

There is provided, in accordance with an example of the present invention, a method for embedding medical additives into a bioabsorbable material. The method may include chemically reacting one or more functional groups of a bioabsorbable material with one or more medical additives. The bioabsorbable material may be configured to be placed inside a body of a human. The one or more medical additives may be configured to remain chemically bonded to a foam structure of the bioabsorbable material until a biodegradation of the bioabsorbable material.

There is provided, in accordance with an example of the present invention, a method for embedding medical additives into a bioabsorbable material. The method may include dissolving one or more water soluble medical additives into an aqueous phase. The method may include incorporating the aqueous phase into a foam network of a bioabsorbable material. The bioabsorbable material may be configured to be placed inside a body of a human.

There is provided, in accordance with an example of the present invention, a bioabsorbable material configured to be used with a surgical staple cartridge configured to repair one or more parts of a body of a human. The bioabsorbable material may have one or more isocyanate groups, and one or more medical additives chemically bonded to the one or more isocyanate groups. The one or more medical additives may include polydrugs.

### BRIEF DESCRIPTION OF THE DRAWINGS

This invention will be more fully understood from the following detailed description taken in conjunction with the accompanying drawings, in which:

FIG. 1 is a perspective view of one exemplary embodiment of a conventional surgical stapling and severing instrument.

FIG. 2A is a top view of a staple cartridge for use with the surgical stapling and severing instrument of FIG. 1;

FIG. 2B is a side view of the staple cartridge of FIG. 2A;

FIG. 3 is a side view of a staple in an unfired (pre-deployed) configuration that can be disposed within the staple cartridge of the surgical cartridge assembly of FIG. 2A;

FIG. 4 is a perspective view of a knife and firing bar (“E-beam”) of the surgical stapling and severing instrument of FIG. 1;

FIG. 5 is a perspective view of a wedge sled of a staple cartridge of the surgical stapling and severing instrument of FIG. 1;

FIG. 6A is a longitudinal cross-sectional view of an exemplary surgical cartridge assembly having a compressible non-fibrous adjunct attached to a top or deck surface of a staple cartridge;

FIG. 6B is a longitudinal cross-sectional view of a surgical end effector having an anvil pivotably coupled to an elongate channel and the surgical cartridge assembly of FIG. 6A disposed within and coupled to the elongate channel, showing the anvil in a closed position without any tissue between the anvil and the adjunct;

FIG. 7 is a partial-schematic illustrating the adjunct of FIGS. 6A-6B in a tissue deployed condition;

FIG. 8 is a perspective view of an exemplary cartridge assembly;

FIG. 9 is a flow chart showing an exemplary method for embedding medical additives into a bioabsorbable material;

FIG. 10 is a flow chart showing an exemplary method for embedding medical additives into a bioabsorbable material.

### DETAILED DESCRIPTION

The following detailed description should be read with reference to the drawings, in which like elements in different drawings are identically numbered. The drawings, which are not necessarily to scale, depict selected embodiments and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±10% of the recited value, e.g., “about 90%” may refer to the range of values from 81% to 99%.

The term “polydrug,” as used herein, refers to a single drug and/or a drug including multiple active pharmaceutical ingredients (APIs) in a single dosage form or implant.

The term “polyurethane,” as used herein, refers to a polymeric reaction product of an isocyanate and a polyol, and is not limited to those polymers which include only urethane or polyurethane linkages. It is well understood by those of ordinary skill in the art of preparing polyurethanes that the polyurethane polymers may also include linkages such as allophanate, carbodiimide, and other linkages described herein in addition to urethane linkages.

The expressions “reaction system,” “reactive formulation,” “reaction product,” and “reactive mixture” are interchangeably used herein, and all refer to a combination of reactive compounds used to make the bioabsorbable material according to the disclosure.

The term “room temperature” refers to temperatures of about 20° C., this means referring to temperatures in the range 18° C. to 25° C. Such temperatures will include, 18° C., 19° C., 20° C., 21° C., 22° C., 23° C., 24° C. and 25° C.

Unless otherwise expressed, the “weight percentage” (indicated as % wt. or wt. %) of a component in a composition refers to the weight of the component over the total weight of the composition in which it is present and is expressed as percentage.

“Glass transition temperature” and “Tg” as referred to herein refers to the temperature at which a reversible transition from a hard glass condition into a rubber-elastic condition occurs.

Surgical stapling assemblies and methods for manufacturing and using the same are provided. In general, a surgical stapling assembly can include a staple cartridge having staples disposed therein and an adjunct configured to be releasably retained on the staple cartridge. As discussed herein, the various adjuncts provided can be configured to compensate for variations in tissue properties, such as variations in tissue thickness, and/or to promote tissue ingrowth when the adjuncts are stapled to tissue. As discussed herein, an adjunct can include a bioabsorbable material, such as a foam.

An exemplary stapling assembly can include a variety of features to facilitate application of a surgical staple, as described herein and illustrated in the drawings. However, a person skilled in the art will appreciate that the stapling assembly can include only some of these features and/or it can include a variety of other features known in the art. The stapling assemblies described herein are merely intended to represent certain exemplary examples. Moreover, while the adjuncts are described in connection with surgical staple cartridge assemblies, the adjuncts can be used in connection with staple reloads that are not cartridge based or any type of surgical instrument.

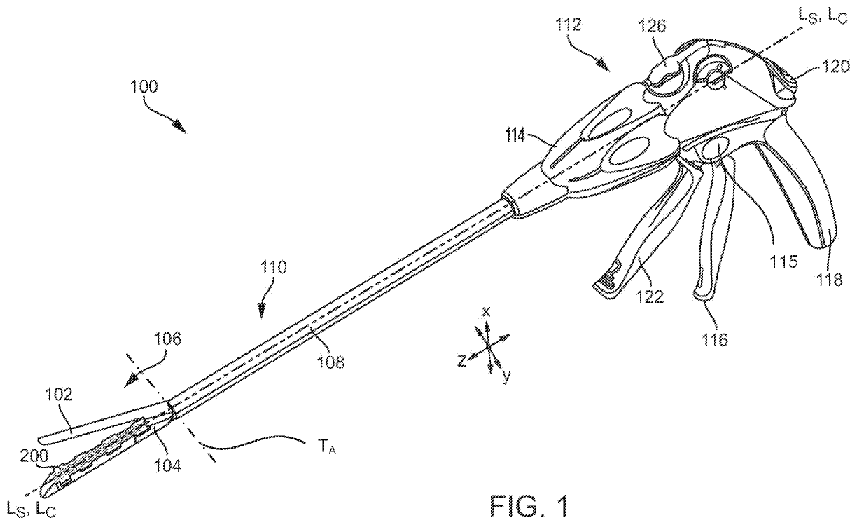

FIG. 1 illustrates an exemplary surgical stapling and severing device 100 suitable for use with an implantable adjunct. The illustrated surgical stapling and severing device 100 includes end effector 106 having an anvil 102 that is pivotably coupled to an elongate channel 104. As a result, the end effector 106 can move between an open position, as shown in FIG. 1, and a closed position in which the anvil 102 is positioned adjacent to the elongate channel 104 to engage tissue therebetween. The end effector 106 can be attached at its proximal end to an elongate shaft 108 forming an implement portion 110. When the end effector 106 is closed, or at least substantially closed, (e.g., the anvil 102 moves from the open position in FIG. 1 toward the elongate channel) the implement portion 110 can present a sufficiently small cross-section suitable for inserting the end effector 106 through a trocar. While the device 100 is configured to staple and sever tissue, surgical devices configured to staple but not sever tissue are also contemplated herein.

In various instances, the end effector 106 can be manipulated by a handle 112 connected to the elongate shaft 108. The handle 112 can include user controls such as a rotation knob 114 that rotates the elongate shaft 108 and the end effector 106 about a longitudinal axis (Ls) of the elongate shaft 108 and an articulation control 115 that can articulate the end effector 106 about an articulate axis (TA) that is substantially transverse to the longitudinal axis (Ls) of the elongate shaft 108. Further controls include a closure trigger 116 which can pivot relative to a pistol grip 118 to close the end effector 106. A closure release button 120 can be outwardly presented on the handle 112 when the closure trigger 116 is clamped such that the closure release button 120 can be depressed to unclamp the closure trigger 116 and open the end effector 106, for example. Handle 112 may also take the form of an interface for connection to a surgical robot.

In some examples, a firing trigger 122, which can pivot relative to the closure trigger 116, can cause the end effector 106 to simultaneously sever and staple tissue clamped therein. The firing trigger 122 may be powered, require force from a user to engage, or some combination thereof. A manual firing release lever 126 can allow the firing system to be retracted before full firing travel has been completed, if desired, and, in addition, the firing release lever 126 can allow a surgeon, or other clinician, to retract the firing system in the event that the firing system binds and/or fails.

Additional details on the surgical stapling and severing device 100 and other surgical stapling and severing devices suitable for use with the present disclosure are described, for example, in U.S. Pat. No. 9,332,984 and in U.S. Patent Publication No. 2009/0090763, the disclosures of which are incorporated herein by reference in their entireties. Further, the surgical stapling and severing device need not include a handle, but instead can have a housing that is configured to couple to a surgical robot, for example, as described in U.S. Patent Publication No. 2019/0059889, the disclosure of which is incorporated herein by reference in its entirety.

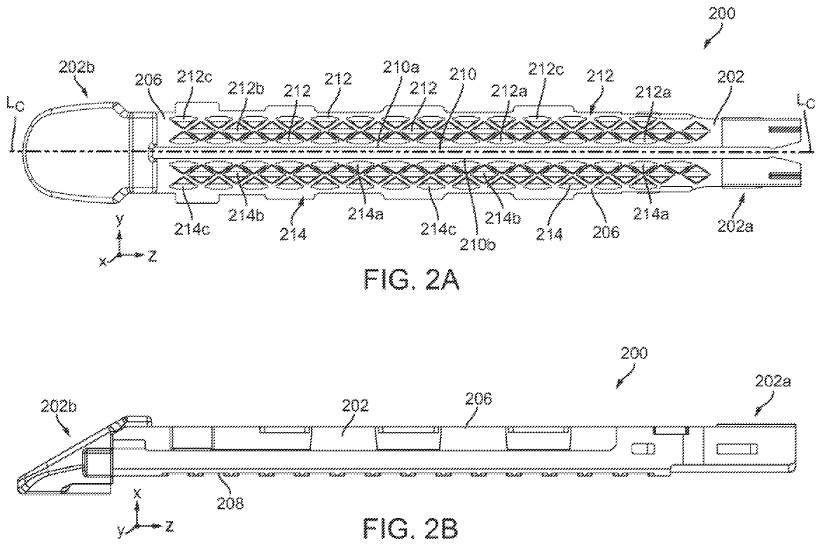

As further shown in FIG. 1, a staple cartridge 200 can be utilized with the instrument 100. In use, the staple cartridge 200 is placed within and coupled to the elongate channel 104. While the staple cartridge 200 can have a variety of configurations, in this illustrated example, the staple cartridge 200, which is shown in more detail in FIGS. 2A-2B, has a proximal end 202a and a distal end 202b with a cartridge longitudinal axis (LC) extending therebetween. As a result, when the staple cartridge 200 is inserted into the elongate channel 104 (FIG. 1), the longitudinal axis (LC) is substantially or approximately parallel with the longitudinal axis (LS) of the elongate shaft 108. Further, the staple cartridge 200 includes a longitudinal slot 210 defined by two opposing walls 210a, 210b and configured to receive at least a portion of a firing member of a firing assembly, like firing assembly 400 in FIG. 4, as discussed further below. As shown, the longitudinal slot 210 extends from the proximal end 202a toward the distal end 202b of the staple cartridge 200. It is also contemplated herein that in other examples, the longitudinal slot 210 can be omitted.

The illustrated staple cartridge 200 includes staple cavities 212, 214 defined therein, in which each staple cavity 212, 214 is configured to removably house at least a portion of a staple (not shown). The number, shape, and position of the staple cavities can vary and can depend at least on the size and shape (e.g., mouth-like shape) of the staples to be removably disposed therein. In this illustrated example, the staple cavities are arranged in two sets of three longitudinal rows, in which the first set of staple cavities 212 is positioned on a first side of the longitudinal slot 210 and the second set of staple cavities 214 is positioned on a second side of the longitudinal slot 210. On each side of the longitudinal slot 210, and thus for each set of rows, a first longitudinal row of staple cavities 212a, 214a extends alongside the longitudinal slot 210, a second row of staple cavities 212b, 214b extends alongside the first row of staple cavities 212a, 214a, and a third row of staple cavities 212c, 214c extends alongside the second row of staple cavities 212b, 214b. Each row may be approximately parallel and the staple cavities that make up the rows may be approximately parallel in orientation with the longitudinal slot 210. As shown in FIGS. 2A, each staple cavity 212, 214 may include a maximum length SL of about 0.122 inches to about 0.124 inches and a maximum width SW of about 0.023 inches to about 0.027 inches. In addition, at least the centers of two adjacent cavities 212, 214 are spaced apart by about 0.158 inches.

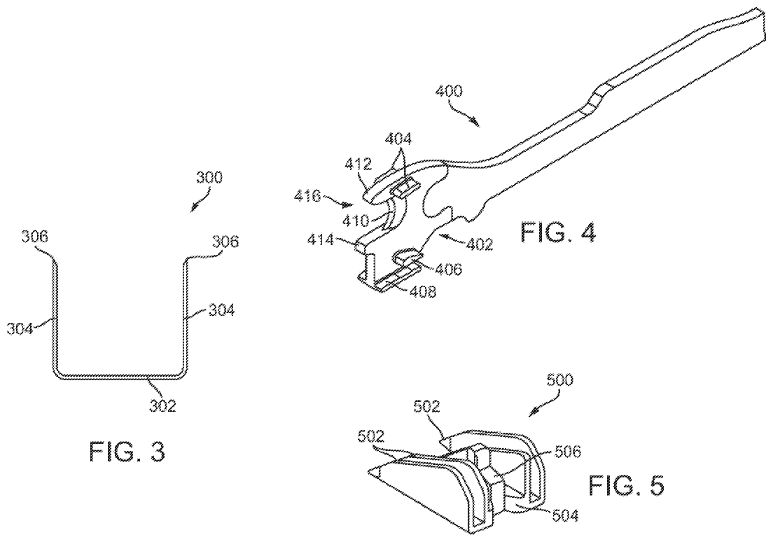

The staples releasably stored in the staple cavities 212, 214 can have a variety of configurations. An exemplary staple 300 that can be releasably stored in each of the staple cavities 212, 214 is illustrated in FIG. 3 in its unfired (pre-deployed, unformed) configuration. The illustrated staple 300 includes a crown (base) 302 and two legs 304 extending from each end of the crown 302. In this example, the crown 302 extends in a linear direction and the staple legs 304 have the same unformed height. Further, prior to the staples 300 being deployed, the staple crowns 302 can be supported by staple drivers that are positioned within the staple cartridge 200 and, concurrently, the staple legs 304 can be at least partially contained within the staple cavities 212, 214. Further, the staple legs 304 can extend beyond a top surface, like top surface 206, of the staple cartridge 200 when the staples 300 are in their unfired positions. In certain instances, as shown in FIG. 3, the tips 306 of the staple legs 304 can be pointed and sharp which can incise and penetrate tissue.

In use, staples 300 can be deformed from an unfired position into a fired position such that the staple legs 304 move through the staple cavities 212, 214, penetrate tissue positioned between the anvil 102 and the staple cartridge 200, and contact the anvil 102. As the staple legs 304 are deformed against the anvil 102, the legs 304 of each staple 300 can capture a portion of the tissue within each staple 300 and apply a compressive force to the tissue. Further, the legs 304 of each staple 300 can be deformed downwardly toward the crown 302 of the staple 300 to form a staple entrapment area in which the tissue can be captured therein. In various instances, the staple entrapment area can be defined between the inner surfaces of the deformed legs and the inner surface of the crown of the staple. The size of the entrapment area for a staple can depend on several factors such as the length of the legs, the diameter of the legs, the width of the crown, and/or the extent in which the legs are deformed, for example.

In some examples, all of the staples disposed within the staple cartridge 200 can have the same unfired (pre-deployed, unformed) configuration. In other examples, the staples can include at least two groups of staples each having a different unfired (pre-deployed, unformed) configuration, e.g., varying in height and/or shape, relative to one another, etc.

Referring back to FIGS. 2A-2B, the staple cartridge 200 extends from a top surface or deck surface 206 to a bottom surface 208, in which the top surface 206 is configured as a tissue-facing surface and the bottom surface 208 is configured as a channel-facing surface. As a result, when the staple cartridge 200 is inserted into the elongate channel 104, as shown in FIG. 1, the top surface 206 faces the anvil 102 and the bottom surface 208 (obstructed) faces the elongate channel 104.

With reference to FIGS. 4 and 5, a firing assembly such as, for example, firing assembly 400, can be utilized with a surgical stapling and severing device, like device 100 in FIG. 1. The firing assembly 400 can be configured to advance a wedge sled 500 having wedges 502 configured to deploy staples from the staple cartridge 200 into tissue captured between an anvil, like anvil 102 in FIG. 1, and a staple cartridge, like staple cartridge 200 in FIG. 1. Furthermore, an E-beam 402 at a distal portion of the firing assembly 400 may fire the staples from the staple cartridge. During firing, the E-beam 402 can also cause the anvil to pivot towards the staple cartridge, and thus move the end effector from the open position towards a closed position. The illustrated E-beam 402 includes a pair of top pins 404, a pair of middle pins 406, which may follow a portion 504 of the wedge sled 500, and a bottom pin or foot 408. The E-beam 402 can also include a sharp cutting edge 410 configured to sever the captured tissue as the firing assembly 400 is advanced distally, and thus towards the distal end of the staple cartridge. In addition, integrally formed and proximally projecting top guide 412 and middle guide 414 bracketing each vertical end of the cutting edge 410 may further define a tissue staging area 416 assisting in guiding tissue to the sharp cutting edge 410 prior to being severed. The middle guide 414 may also serve to engage and fire the staples within the staple cartridge by abutting a stepped central member 506 of the wedge sled 500 that effects staple formation by the end effector 106.

In use, the anvil 102 in FIG. 1 can be moved into a closed position by depressing the closure trigger in FIG. 1 to advance the E-beam 402 in FIG. 4. The anvil 102 can position tissue against at least the top surface 206 of the staple cartridge 200 in FIGS. 2A-2B. Once the anvil has been suitably positioned, the staples 300 in FIG. 3 disposed within the staple cartridge can be deployed.

To deploy staples from the staple cartridge, as discussed above, the sled 500 in FIG. 5 can be moved from the proximal end toward a distal end of the cartridge body, and thus, of the staple cartridge. As the firing assembly 400 in FIG. 4 is advanced, the sled can contact and lift staple drivers within the staple cartridge upwardly within the staple cavities 212, 214. In at least one example, the sled and the staple drivers can each include one or more ramps, or inclined surfaces, which can co-operate to move the staple drivers upwardly from their unfired positions. As the staple drivers are lifted upwardly within their respective staple cavities, the staples are advanced upwardly such that the staples emerge from their staple cavities and penetrate into tissue. In various instances, the sled can move several staples upwardly at the same time as part of a firing sequence.

As indicated above, the stapling device can be used in combination with a compressible adjunct. A person skilled in the art will appreciate that, while adjuncts are shown and described below, the adjuncts disclosed herein can be used with other surgical instruments and need not be coupled to a staple cartridge as described. Further, a person skilled in the art will also appreciate that the staple cartridges need not be replaceable.

As discussed above, with some surgical staplers, a surgeon is often required to select the appropriate staples having the appropriate staple height for tissue to be stapled. For example, a surgeon will utilize tall staples for use with thick tissue and short staples for use with thin tissue. In some instances, however, the tissue being stapled does not have a consistent thickness and thus, the staples cannot achieve the desired fired configuration for every section of the stapled tissue (e.g., thick and thin tissue sections). The inconsistent thickness of tissue can lead to undesirable leakage and/or tearing of tissue at the staple site when staples with the same or substantially greater height are used, particularly when the staple site is exposed to intra-pressures at the staple site and/or along the staple line.

Accordingly, various examples of adjuncts are provided that can be configured to compensate for varying thickness of tissue that is captured within fired (deployed) staples to avoid the need to take into account staple height when stapling tissue during surgery. That is, the adjuncts described herein can allow a set of staples with the same or similar heights to be used in stapling tissue of varying thickness (e.g., from thin to thick tissue) while also, in combination with the adjunct, providing adequate tissue compression within and between fired staples. Thus, the adjuncts described herein can maintain suitable compression against thin or thick tissue stapled thereto to thereby minimize leakage and/or tearing of tissue at the staple sites. In addition, exemplary adjuncts described herein may be configured to be absorbed in the body over a period of 100 to 300 days depending on implanted location and tissue health.

Alternatively, or in addition, the adjuncts can be configured to promote tissue ingrowth. In various instances, it is desirable to promote the ingrowth of tissue into an implantable adjunct, to promote the healing of the treated tissue (e.g., stapled and/or incised tissue), and/or to accelerate the patient's recovery. More specifically, the ingrowth of tissue into an implantable adjunct may reduce the incidence, extent, and/or duration of inflammation at the surgical site. Tissue ingrowth into and/or around the implantable adjunct may, for example, manage the spread of infections at the surgical site. The ingrowth of blood vessels, especially white blood cells, for example, into and/or around the implantable adjunct may fight infections in and/or around the implantable adjunct and the adjacent tissue. Tissue ingrowth may also encourage the acceptance of foreign matter (e.g., the implantable adjunct and the staples) by the patient's body and may reduce the likelihood of the patient's body rejecting the foreign matter. Rejection of foreign matter may cause infection and/or inflammation at the surgical site.

In general, the adjuncts provided herein are designed and positioned atop a staple cartridge, like staple cartridge 200. When the staples are fired (deployed) from the cartridge, the staples penetrate through the adjunct and into tissue. As the legs of the staple are deformed against the anvil that is positioned opposite the staple cartridge, the deformed legs capture a portion of the adjunct and a portion of the tissue within each staple. That is, when the staples are fired into tissue, at least a portion of the adjunct becomes positioned between the tissue and the fired staple. While the adjuncts described herein can be configured to be attached to a staple cartridge, it is also contemplated herein that the adjuncts can be configured to mate with other instrument components, such as an anvil of a surgical stapler. A person of ordinary skill will appreciate that the adjuncts provided herein can be used with replaceable cartridges or staple reloads that are not cartridge based.

### Methods of Stapling Tissue

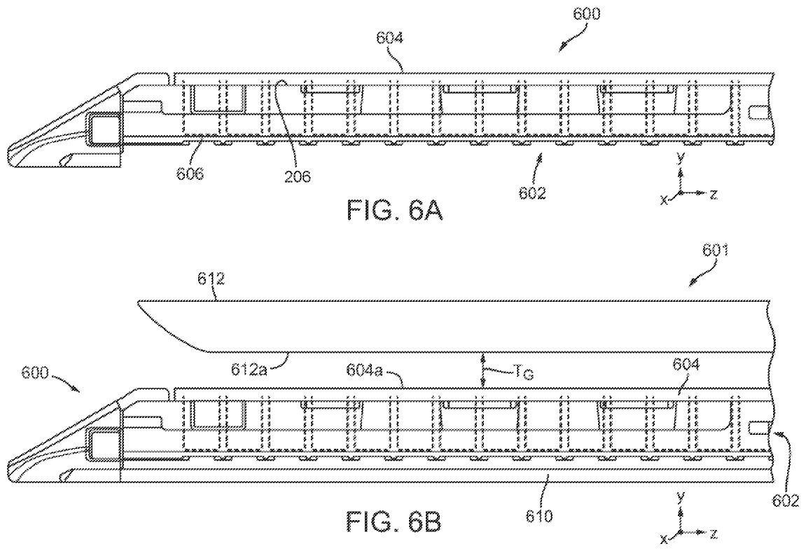

FIGS. 6A-6B illustrate an exemplary example of a stapling assembly 600 that includes a staple cartridge 200 and an adjunct 604. For sake of simplicity, the adjunct 604 is generally illustrated in FIGS. 6A-6B, and various configurations of the adjunct are described in more detail below. As shown, the adjunct 604 is positioned against the staple cartridge 200. While partially obstructed in FIGS. 6A-6B, the staple cartridge 200 includes staples 300, that are configured to be deployed into tissue. The staples 300 can have any suitable unformed (pre-deployed) height.

In the illustrated example, the adjunct 604 can be mated to at least a portion of the top surface or deck surface 206 of the staple cartridge 602. In some examples, the top surface 206 of the staple cartridge 200 can include one or more surface features which can be configured to engage the adjunct 604 to avoid undesirable movements of the adjunct 604 relative to the staple cartridge 200 and/or to prevent premature release of the adjunct 604 from the staple cartridge 200. Exemplary surface features are described further below and in U.S. Patent Publication No. 2016/0106427, which is incorporated by reference herein in its entirety.

FIG. 6B shows the stapling assembly 600 placed within and coupled to the elongate channel 610 of surgical end effector 106. The anvil 102 is pivotally coupled to the elongate channel 610 and is thus moveable between open and closed positions relative to the elongate channel 610, and thus the staple cartridge 200. The anvil 102 is shown in a closed position in FIG. 6B and illustrates a tissue gap T_{G1} created between the staple cartridge 602 and the anvil 612. More specifically, the tissue gap T_{G1} is defined by the distance between the tissue-compression surface 102a of the anvil 102 (e.g., the tissue-engaging surface between staple forming pockets in the anvil) and the tissue-contacting surface 604a of the adjunct 604. In this illustrated example, both the tissue-compression surface 102a of the anvil 102 and the tissue-contacting surface 604a of the adjunct 604 is planar, or substantially planar (e.g., planar within manufacturing tolerances). As a result, when the anvil 102 is in a closed position, as shown in FIG. 6B, the tissue gap T_{G1} is generally uniform (e.g., nominally identical within manufacturing tolerances) when no tissue is disposed therein. In other words, the tissue gap T_{G1} is generally constant (e.g., constant within manufacturing tolerances) across the end effector 106 (e.g., in the y-direction). In other examples, the tissue-compression surface of the anvil can include a stepped surface having longitudinal steps between adjacent longitudinal portions, and thus create a stepped profile (e.g., in the y-direction). In such examples, the tissue gap T_{G1} can be varied.

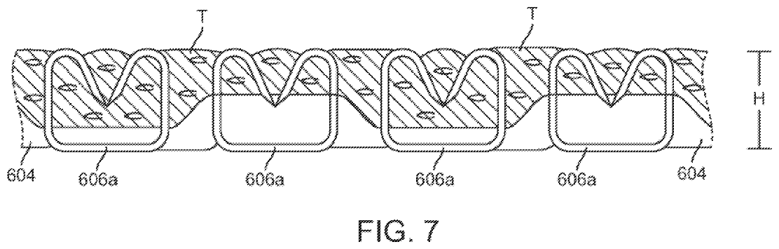

The adjunct 604 is compressible to permit the adjunct to compress to varying heights to thereby compensate for different tissue thickness that are captured within a deployed staple. The adjunct 604 has an uncompressed (undeformed), or pre-deployed, height and is configured to deform to one of a plurality of compressed (deformed), or deployed, heights. For example, the adjunct 604 can have an uncompressed height which is greater than the fired height of the staples 300 disposed within the staple cartridge 200 (e.g., the height (H) of the fired staple 300a in FIG. 7). That is, the adjunct 604 can have an undeformed state in which a maximum height of the adjunct 604 is greater than a maximum height of a fired staple (e.g., a staple that is in a formed configuration).

In use, once the surgical stapling and severing device, like device 100 in FIG. 1, is directed to the surgical site, tissue is positioned between the anvil 102 and the stapling assembly 600 such that the anvil 102 is positioned adjacent to a first side of the tissue and the stapling assembly 600 is positioned adjacent to a second side of the tissue (e.g., the tissue can be positioned against the tissue-contacting surface 604a of the adjunct 604). Once tissue is positioned between the anvil 102 and the stapling assembly 600, the surgical stapler can be actuated, e.g., as discussed above, to thereby clamp the tissue between the anvil 102 and the stapling assembly 600 (e.g., between the tissue-compression surface 102a of the anvil 102 and the tissue-contacting surface 604a of the adjunct 604) and to deploy staples from the cartridge through the adjunct and into the tissue to staple and attach the adjunct to the tissue.

As shown in FIG. 7, when the staples 300 are fired, tissue (T) and a portion of the adjunct 604 are captured by the fired (formed) staples 300a. The fired staples 300a each define the entrapment area therein, as discussed above, for accommodating the captured adjunct 604 and tissue (T). The entrapment area defined by a fired staple 300a is limited, at least in part, by a height (H) of the fired staple 300a.

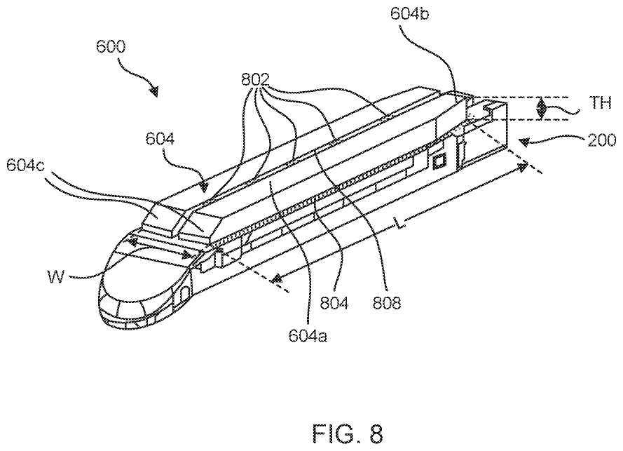

FIG. 8 illustrates a perspective view of a staple cartridge assembly 600 with an adjunct 604 and a staple cartridge 200. The adjunct 604 has a tissue contacting surface 604a, a proximal end 604a, and a distal end 604b. The adjunct 604 may include a slot/slit 808 separating or partially separating two parallel portions of the adjunct 604. In one example, adjunct 604 may include a slot 808 separating two parallel portions of the adjunct 604, while in another example, adjunct 604 may include a slit 808 separating two parallel portions of the adjunct 604 and also one or more bridges (e.g., five bridges) 802 connecting the two parallel portions of the adjunct 604. At least one bridge has a length in the longitudinal direction of about 0.035 inches to about 0.046 inches. The adjunct 604 has a length L of about 40 mm to about 80 mm, such as about 60 mm to about 65 mm, about 66.04 mm to about 66.3 mm, about 45 mm to about 55 mm, or about 51.12 mm to about 51.38 mm. The adjunct 604 has a width W of about 8 mm to about 12 mm, such as about 9.75 mm to about 10.25 mm or about 10.025 mm to about 10.035 mm. The adjunct 604 may also have a thickness or height TH of about 2.5 mm to about 3.5 mm, such as about 2.85 mm to about 3.15 mm or about 2.95 mm to about 3.05 mm.

The cartridge 200 has a height CH of about 6.3 mm to about 8.1 mm, a width CW of about 8.9 mm to about 14 mm, and a length CL of about 80 to about 90 mm such as about 86.7 mm.

The staple cartridge 200 may include one or more raised ledges 804 along one or more sides of the adjunct 602 to help align the adjunct 604 on the deck of the staple cartridge 200.

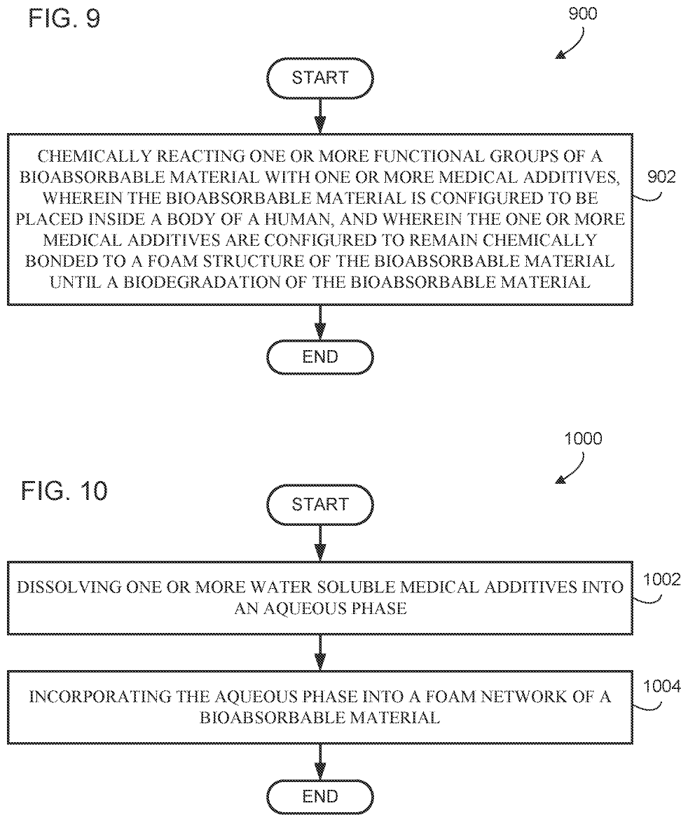

Referring to FIGS. 9-10, a surgical adjunct 604, including a bioabsorbable material that itself includes a foam, may be embedded with one or more medical additives using method 900 (FIG. 9) or method 1000 (FIG. 10). The embedding techniques described herein may offer an added benefit of increasing positive in vivo interactions, including biocompatibility, wound healing, tissue integration, chemotherapy, anti-inflammatory, bone growth and integration, ligament and tendon repair, etc., when the adjunct is delivered to the tissue site, as described herein. As such, the embedding techniques described herein may allow the bioabsorbable material itself to aid in the healing process of the surrounding tissue. In addition, the embedding techniques described herein may offer an added benefit of preventing fibrous encapsulation of the foam cushion, and/or providing tunable release profiles for a variety of medical additives delivered to the tissue site.

Specifically with respect to FIG. 9, the method 900 used for embedding medical additives into a bioabsorbable material (e.g., a foam) may include chemically reacting one or more functional groups of the bioabsorbable material with one or more medical additives (step 902). Chemical embedment may include introducing the medical additive(s) through chemical reaction and bonding with a foam structure of the bioabsorbable material, such that the bioabsorbable material may maintain a slow and steady release profile as it degrades over time.

In some examples, the bioabsorbable material may include a polyurethane and/or be a reaction product of a polyol and an isocyanate. In such examples, the medical additive(s) used may react with the polyol.

In some examples, the functional group(s) may include isocyanate groups. In such examples, the medical additive(s) may include polydrugs (e.g., ibuprofen, acetaminophen, naproxen, etc.), and the method may include endcapping the isocyanate reactive groups in the prepolymer with the polydrug(s) such that the polydrug(s) remain chemically bonded to a foam structure of the bioabsorbable material until biodegradation. The polydrug(s) may be configured to treat pain and/or promote wound healing, tissue growth, infection reduction, localized radiotherapeutics, immunotherapies, and the like.

In some examples, the functional group(s) may include polyol hydroxyl groups (e.g., polyethylene glycol modified (PEGylated) biopharmaceuticals), such as ones that may be incorporated as biobetters. In such examples, the medical additive(s) may include medications that treat pain and/or promote wound healing, tissue growth, infection reduction, and the like. The method may include chemically reacting the polyol hydroxyl groups with the medical additive(s) by functionalizing the polyol hydroxyl groups with the medical additive(s). The method may include chemically reacting the polyol hydroxyl groups with one or more natural materials, such as alginate, hyaluronic acid, etc.

In some examples, chemically reacting the functional groups of the bioabsorbable material may include modifying the functional groups using one or more reactive surfactants with enhanced polyether modifications, such as incorporating PEGylated biopharmaceuticals as biobetters.

Specifically with respect to FIG. 10, the method 1000 used for embedding medical additives into a bioabsorbable material (e.g., a foam) may include physical embedment of medical additives into the bioabsorbable material, which may involve introducing the medical additives into the bioabsorbable material as independent substances which do not themselves react chemically with the system. As shown, method 1000 may involve dissolving one or more water soluble medical additives into an aqueous phase (step 1002), and incorporating the aqueous phase into a foam network of the bioabsorbable material (step 1004). In some examples, the medical additives may include water soluble drugs and/or vitamins, such as antihypertensive drugs or B-complex vitamins. These drugs and/or vitamins can be incorporated into a foam network of the bioabsorbable material during processing and released during biodegradation of the bioabsorbable material after delivery to the tissue.

In some examples, dissolving the water soluble medical additives into the aqueous phase may involve generating a colloidal suspension including silver nanoparticles. This process may allow for off-gassing produced by the water, while keeping the silver nanoparticles embedded in the bioabsorbable material. This process may provide an added benefit of enhanced wound healing due to the significant antimicrobial effects of silver nanoparticles.

In some examples, the method may include an emulsion method whereby the medical additives may be encapsulated within “shells” or areas of the bioabsorbable material cells, and released during biodegradation of the bioabsorbable material. For example, the medical additives may be encapsulated within the bioabsorbable material via in-situ hydrogel formation (e.g., entrapment within the bioabsorbable material cells after the adjunct is positioned and exposed to water within the body, etc.).

As will be appreciated by one skilled in the art, The embodiments described above are cited by way of example, and the present invention is not limited by what has been particularly shown and described hereinabove. Rather, the scope of the invention includes both combinations and sub combinations of the various features described hereinabove, as well as variations and modifications thereof which would occur to persons skilled in the art upon reading the foregoing description and which are not disclosed in the prior art.

In some examples, disclosed devices (e.g., end effector, surgical adjunct, and/or staple cartridges) and methods involving one or more disclosed devices may involve one or more of the following clauses:

Clause 1: A method for embedding medical additives into a bioabsorbable material, the method comprising: chemically reacting one or more functional groups of a bioabsorbable material with one or more medical additives, wherein the bioabsorbable material is configured to be placed inside a body of a human, and wherein the one or more medical additives are configured to remain chemically bonded to a foam structure of the bioabsorbable material until a biodegradation of the bioabsorbable material.

Clause 2: The method of clause 1, wherein the bioabsorbable material comprises polyurethane.

Clause 3: The method of clause 2, wherein the bioabsorbable material is a reaction product of a polyol and an isocyanate.

Clause 4: The method of clause 3, wherein the one or more medical additives react with the polyol.

Clause 5: The method of clause 1, wherein the bioabsorbable material is configured to be used with a surgical staple cartridge configured to repair one or more parts of the body of the human.

Clause 6: The method of clause 1, wherein the one or more functional groups comprise isocyanate groups.

Clause 7: The method of clause 6, wherein the one or more medical additives comprise one or more polydrugs.

Clause 8: The method of clause 7, wherein chemically reacting the one or more functional groups of the bioabsorbable material with the one or more medical additives comprises endcapping the isocyanate groups with the one or more polydrugs.

Clause 9: The method of clause 7, wherein the polydrugs treat one or more of pain, wound healing, tissue growth, infection reduction, immunosuppression, radiotherapy, or combinations thereof.

Clause 10: The method of clause 1, wherein the one or more functional groups comprise polyol hydroxyl groups.

Clause 11: The method of clause 10, wherein chemically reacting the one or more functional groups of the bioabsorbable material with the one or more medical additives comprises functionalizing the polyol hydroxyl groups with the one or more medical additives.

Clause 12: The method of clause 11, wherein the one or more medical additives comprise one or more medications that treat one or more of pain, wound healing, tissue growth, infection reduction, immunosuppression, radiotherapy, or combinations thereof.

Clause 13: The method of clause 1, wherein chemically reacting the one or more functional groups of the bioabsorbable material with the one or more medical additives comprises modifying the one or more functional groups via one or more reactive surfactants.

Clause 14: A method for embedding medical additives into a bioabsorbable material, the method comprising: dissolving one or more water soluble medical additives into an aqueous phase; and incorporating the aqueous phase into a foam network of a bioabsorbable material, wherein the bioabsorbable material is configured to be placed inside a body of a human.

Clause 15: The method of clause 14, wherein the one or more water soluble medical additives are configured to be released into the body of the human over a period of time during a biodegradation of the bioabsorbable material.

Clause 16: The method of clause 14, wherein dissolving the one or more water soluble medical additives into the aqueous phase comprises generating a colloidal suspension comprising one or more silver nanoparticles.

Clause 17: The method of clause 14, wherein the one or more water soluble medical additives comprise at least one of drugs or vitamins.

Clause 18: A bioabsorbable material configured to be used with a surgical staple cartridge configured to repair one or more parts of a body of a human, the bioabsorbable material comprising: one or more isocyanate groups; and one or more medical additives chemically bonded to the one or more isocyanate groups, wherein the one or more medical additives comprise polydrugs.

Clause 19: The bioabsorbable material of clause 18, wherein the one or more medical additives are configured to remain chemically bonded to a foam structure of the bioabsorbable material until a biodegradation of the bioabsorbable material.

Clause 20: The bioabsorbable material of clause 18, wherein the polydrugs treat one or more of pain, wound healing, tissue growth, infection reduction, immunosuppression, radiotherapy, or combinations thereof.

Clause 21: A method for embedding medical additives into a bioabsorbable material, the method comprising: chemically reacting one or more functional groups of a bioabsorbable material with one or more medical additives, wherein the bioabsorbable material is configured to be placed inside a body of a human, and wherein the one or more medical additives are configured to remain chemically bonded to a foam structure of the bioabsorbable material until a biodegradation of the bioabsorbable material.

Clause 22: The method of clause 21, wherein the bioabsorbable material comprises polyurethane.

Clause 23: The method of any of clauses 21-22, wherein the bioabsorbable material is a reaction product of a polyol and an isocyanate.

Clause 24: The method of clause 23, wherein the one or more medical additives react with the polyol.

Clause 25: The method of any of clauses 21-24, wherein the bioabsorbable material is configured to be used with a surgical staple cartridge configured to repair one or more parts of the body of the human.

Clause 26: The method of any of clauses 21-25, wherein the one or more functional groups comprise isocyanate groups.

Clause 27: The method of any of clauses 21-26, wherein the one or more medical additives comprise one or more polydrugs.

Clause 28: The method of clause 27, wherein chemically reacting the one or more functional groups of the bioabsorbable material with the one or more medical additives comprises endcapping the isocyanate groups with the one or more polydrugs.

Clause 29: The method of any of clauses 27-28, wherein the polydrugs treat one or more of pain, wound healing, tissue growth, infection reduction, or combinations thereof.

Clause 30: The method of any of clauses 21-25, wherein the one or more functional groups comprise polyol hydroxyl groups.

Clause 31: The method of clause 30, wherein chemically reacting the one or more functional groups of the bioabsorbable material with the one or more medical additives comprises functionalizing the polyol hydroxyl groups with the one or more medical additives.

Clause 32: The method of any of clauses 21-31, wherein the one or more medical additives comprise one or more medications that treat one or more of pain, wound healing, tissue growth, infection reduction, or combinations thereof.

Clause 33: The method of any of clauses 21-32, wherein chemically reacting the one or more functional groups of the bioabsorbable material with the one or more medical additives comprises modifying the one or more functional groups via one or more reactive surfactants.

Clause 34: The method of any of clauses 21-33, wherein chemically reacting the one or more functional groups of the bioabsorbable material with the one or more medical additives allows for a steady release profile of the one or more medical additives from the foam structure during the biodegradation of the bioabsorbable material.

Clause 35: The method of any of clauses 21-34, wherein the one or more medical additives treat one or more of pain, wound healing, tissue growth, infection reduction, or combinations thereof.

1-15. (canceled)

16. A method for embedding medical additives into a bioabsorbable material, the method comprising:
chemically reacting one or more functional groups of a bioabsorbable material with one or more medical additives, wherein the bioabsorbable material is configured to be placed inside a body of a human, and
wherein the one or more medical additives are configured to remain chemically bonded to a foam structure of the bioabsorbable material until a biodegradation of the bioabsorbable material.

17. The method of claim 16, wherein the bioabsorbable material comprises polyurethane.

18. The method of claim 17, wherein the bioabsorbable material is a reaction product of a polyol and an isocyanate.

19. The method of claim 18, wherein the one or more medical additives react with the polyol.

20. The method of claim 16, wherein the bioabsorbable material is configured to be used with a surgical staple cartridge configured to repair one or more parts of the body of the human.

21. The method of claim 16, wherein the one or more functional groups comprise isocyanate groups.

22. The method of claim 21, wherein the one or more medical additives comprise one or more polydrugs.

23. The method of claim 22, wherein chemically reacting the one or more functional groups of the bioabsorbable material with the one or more medical additives comprises endcapping the isocyanate groups with the one or more polydrugs.

24. The method of claim 22, wherein the polydrugs treat one or more of pain, wound healing, tissue growth, infection reduction, immunosuppression, radiotherapy, or combinations thereof.

25. The method of claim 16, wherein the one or more functional groups comprise polyol hydroxyl groups.

26. The method of claim 25, wherein chemically reacting the one or more functional groups of the bioabsorbable material with the one or more medical additives comprises functionalizing the polyol hydroxyl groups with the one or more medical additives.

27. The method of claim 26, wherein the one or more medical additives comprise one or more medications that treat one or more of pain, wound healing, tissue growth, infection reduction, immunosuppression, radiotherapy, or combinations thereof.

28. The method of claim 16, wherein chemically reacting the one or more functional groups of the bioabsorbable material with the one or more medical additives comprises modifying the one or more functional groups via one or more reactive surfactants.

29. A method for embedding medical additives into a bioabsorbable material, the method comprising:
dissolving one or more water soluble medical additives into an aqueous phase; and incorporating the aqueous phase into a foam network of a bioabsorbable material,
wherein the bioabsorbable material is configured to be placed inside a body of a human.

30. The method of claim 29, wherein the one or more water soluble medical additives are configured to be released into the body of the human over a period of time during a biodegradation of the bioabsorbable material.

31. The method of clause 29, wherein dissolving the one or more water soluble medical additives into the aqueous phase comprises generating a colloidal suspension comprising one or more silver nanoparticles.

32. The method of clause 29, wherein the one or more water soluble medical additives comprise at least one of drugs or vitamins.

33. A bioabsorbable material configured to be used with a surgical staple cartridge configured to repair one or more parts of a body of a human, the bioabsorbable material comprising: one or more isocyanate groups; and one or more medical additives chemically bonded to the one or more isocyanate groups, wherein the one or more medical additives comprise polydrugs.

34. The bioabsorbable material of claim 33, wherein the one or more medical additives are configured to remain chemically bonded to a foam structure of the bioabsorbable material until a biodegradation of the bioabsorbable material.

35. The bioabsorbable material of claim 33, wherein the polydrugs treat one or more of pain, wound healing, tissue growth, infection reduction, immunosuppression, radiotherapy, or combinations thereof.
